(19) United States
(12) Patent Application Publication
(10) Pub. No.: US 2017/0152509 A1
(43) Pub. Date: Jun. 1, 2017

(54) METHOD FOR OPERATING MAGNETIC BODY PARTICLES AND DEVICE FOR OPERATING MAGNETIC BODY PARTICLES

(71) Applicant: Shimadzu Corporation, Kyoto (JP)

(72) Inventor: Tetsuo OHASHI, Kyoto (JP)

(73) Assignee: Shimadzu Corporation, Kyoto (JP)

(21) Appl. No.: 15/313,662

(22) PCT Filed: May 23, 2014
(86) PCT No.: PCT/JP2014/063747

§ 371 Date: Nov. 23, 2016

(51) Int. Cl. C12N 15/10; G01N 1/40; B03C 1/01; C12Q 1/68

(52) U.S. Cl. CPC C12N 15/1013; C12Q 1/6806; G01N 1/405; B03C 1/01; G01N 2001/4038

(57) ABSTRACT

A method for operating magnetic body particles that is for fixing a target substance in a liquid sample to the surface of the magnetic body particles; and a device for operating magnetic body particles that is used for said method. The magnetic body particles are particles to which a target substance can be fixed selectively. In this method, in a state where a liquid sample, the magnetic body particles, and a magnetic solid body that has a larger particle diameter than that of the magnetic body particle are made to coexist in a container, the magnetic body particles are moved together with the magnetic solid body within the liquid sample by operating a magnetic field from outside the container. By this operation, the target substance can be fixed selectively to the surface of the magnetic body particles.

### TECHNICAL FIELD

The present invention relates to a method for operating magnetic body particles whereby a target substance in a sample is selectively bound to surfaces of the magnetic body particles. The present invention also relates to a device for operating magnetic body particles for use in the method.

### BACKGROUND ART

For the purpose of medical testing, control of food safety and health, or monitoring for environmental protection, there is a need to extract a target substance from a sample containing a wide range of contaminants, and use it for detection or reaction. For example, genetic testing requires efficiently extracting DNA or RNA from biological samples such as the blood, serum, cells, urine, and feces of animals and plants, and viruses before amplification of the target nucleic acid by a method such as PCR.

For the extraction and purification of a target substance in a sample, a method has been developed and is available that uses magnetic body particles measuring about 0.5 μm to less than 20 μm in particle size and having a surface with chemical affinity to the target substance or with a molecular recognition function for recognizing the target substance. In such a method, the following procedure is repeatedly performed which includes: separating and collecting the magnetic body particles from a liquid phase by a magnetic field procedure after the target substance is bound to the surfaces of the magnetic body particles; after optionally dispersing the collected magnetic body particles in a liquid phase such as a washing liquid, separating and collecting from the magnetic body particles the liquid phase. Upon dispersing the magnetic body particles in an elution liquid thereafter, the target substance bound to the magnetic body particles becomes liberated in the elution liquid, and collected from the elution liquid. The magnetic body particles enable magnetic collection of a target substance, which makes centrifugation unnecessary, and therefore, the method is advantageous for automation of chemical extraction and purification.

Magnetic body particles capable of selectively binding a target substance are commercially available as a component of a separation and purification kit. Such kits come with a plurality of reagents in separate containers, and a user measures and dispenses the reagents with a pipette or the like. A device that performs such a pipetting procedure or magnetic field procedure in automation is also commercially available (for example, PTL 1). As an alternative to the pipetting procedure, a method is proposed that separates and purifies a target substance by allowing magnetic body particles to move along the tube length of a tubular device that includes alternately disposed aqueous liquid layers such as a lysis/binding liquid, a washing liquid, and an elution liquid, and gelatinous medium layers (for example, PTL 2). In such a tubular device, a series of procedures can be performed in a sealed system, and therefore, the risk of contamination is smaller than in the open system of the pipetting procedure.

Regardless of whether the pipetting procedure or the procedure using a device with a sealed gel is used, the separation and purification by magnetic body particles involves lysing a biological sample, and binding a target substance, such as nucleic acids, to surfaces of the magnetic body particles. The lysis/binding step requires selectively binding the target substance in a liquid sample to surfaces of the magnetic body particles. A biological sample contains a wide range of contaminants other than the target substance, and when the contaminants become attached to the surfaces of the magnetic body particles binding of the target substance to the magnetic body particles is inhibited, and the collection rate of the target substance drops. For example, in nucleic acid extraction from blood, contaminating proteins from cells may attach to agglomerate on the surfaces of the magnetic body particles, and inhibit binding of nucleic acids to the magnetic body particles.

In order to prevent this, a protein degrading enzyme such as proteinase K is typically added to a sample before the sample is brought into contact with magnetic body particles, and an enzyme treatment is performed under applied heat of 50° C. to 70° C. to decompose and remove the proteins that bind to nucleic acids. The magnetic body particles are added after making the liquid sample more hydrophobic by addition of an alcohol such as ethanol. This enables selective binding of nucleic acids to the surfaces of the magnetic body particles.

Because alcohols inhibit enzyme reaction, an alcohol needs to be added after performing an enzyme treatment under heated conditions when an enzyme treatment is performed in the lysis/binding step. The magnetic body particles need to be added after the enzyme treatment because the contaminating proteins attach to the magnetic body particles, and mask the particle surface when the magnetic body particles are added to the sample before enzyme treatment.

### CITATION LIST

### Patent Literature

### PTL 1: WO97/44671

### PTL 2: WO2012/086243

### SUMMARY OF INVENTION

### Technical Problem

As described above, the enzyme, the magnetic body particles, and an alcohol need to be stored in separate containers, or these need to be separated from one another with dividing walls or other means provided in a container, and added to a sample in series for the separation and purification procedure when performing an enzyme treatment for the lysis/binding process in the separation and purification procedure using magnetic body particles. This complicates the lysis/binding procedure, or requires intricate processes for providing dividing walls or other structures in the device. Either way, the manufacturing cost of the device increases, which is a problem.

The serial addition of an enzyme and magnetic body particles to the device requires adding these components in an open system. This increases the contamination risk even with a device including gel layers and liquid layers disposed therein such as that disclosed in PTL 2. Further, because the enzyme easily becomes deactivated at ordinary temperature, it needs to be stored in a refrigerator or a freezer until the separation and purification procedure. Since the enzyme itself is expensive, the method using an enzyme for lysis and binding is not suited for easy devices intended to process large numbers of samples.

It is accordingly an object of the present invention to provide a method for operating magnetic body particles whereby a target substance can be efficiently bound to surfaces of magnetic body particles with a simple procedure in the separation and purification of the target substance using magnetic body particles, without an enzyme treatment.

### Solution to Problem

Studies by the present inventor found that a target substance such as nucleic acids can be efficiently bound to surfaces of magnetic body particles when a magnetic field procedure is performed in the presence of a magnetic solid body having a larger particle diameter than the magnetic body particles, and the magnetic body particles are dispersed in a liquid with the movement of the magnetic solid body. The present invention was completed on the basis of this finding.

The present invention is concerned with a method for operating magnetic body particles whereby a target substance in a liquid sample is bound to surfaces of magnetic body particles, and a device for operating magnetic body particles for use in the method. The magnetic body particles are particles capable of selectively binding a target substance. Examples of target substances that can selectively bind to the magnetic body particles include biological samples such as nucleic acids, proteins, sugars, lipids, antibodies, receptors, antigens, ligands, and cells.

In a method of the present invention, a magnetic field procedure is performed from outside a container in the presence of a liquid sample, magnetic body particles, and a magnetic solid body having a larger particle diameter than the magnetic body particles in the container to move the magnetic body particles with the magnetic solid body in the liquid sample. For example, the magnetic body particles move back and forth in the liquid sample with the magnetic solid body following the reciprocating movement of a magnet along the outer wall surface of the container. As a result of this procedure, the target substance can selectively bind to the surfaces of the magnetic body particles. In an embodiment of the present invention, the liquid sample contains a component that can lyse cells, such as a chaotropic substance, and a surfactant.

The magnetic solid body is preferably one having a particle diameter of 50 μm or more. Preferably, the particle diameter of the magnetic solid body is at least 10 times larger than the particle diameter of the magnetic body particles. The magnetic solid body may have a surface coating layer to prevent corrosion in the liquid.

In an embodiment of the present invention, the magnetic body particles with the attached target substance are brought into contact with an elution liquid after the target substance has selectively bound to the surfaces of the magnetic body particles according to the foregoing method. This causes the target substance to elute into the elution liquid, and the target substance can be collected.

In a device for operating magnetic body particles of the present invention, the liquid sealed inside a container contains the magnetic body particles capable of selectively binding a target substance, together with the magnetic solid body having a larger particle diameter than the magnetic body particles. In an embodiment, the liquid sealed inside the container is a liquid capable of lysing cells.

### Advantageous Effects of Invention

The method of the present invention efficient disperses magnetic body particles by performing a magnetic field procedure in the presence of the magnetic body particles and the magnetic solid body in a liquid sample containing a target substance. This enables the target substance in the liquid sample to efficiently bind to surfaces of the magnetic body particles, even without an enzyme treatment using an enzyme such as protease. The application of present invention to, for example, separation and purification of a target substance such as nucleic acids enables collection of a high-purity target substance in high yield.

### BRIEF DESCRIPTION OF DRAWINGS

FIG. 1 shows diagrams schematically representing an outline of a method for operating magnetic body particles.

FIG. 2-1 shows diagrams schematically representing the nucleic acid separation and purification steps of an embodiment.

FIG. 2-2 shows diagrams schematically representing the nucleic acid separation and purification steps of an embodiment.

FIG. 3 shows UV absorption spectra of nucleic acids extracted and purified with the magnetic body particle procedure of Example, Reference Example, and Comparative Example.

### DESCRIPTION OF EMBODIMENTS

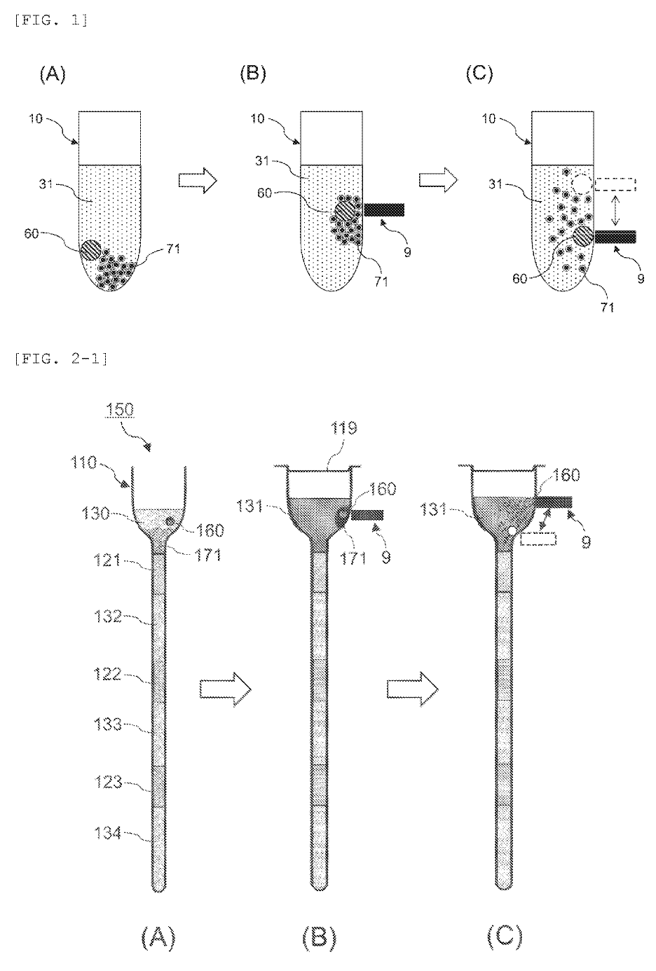

FIG. 1 is a schematic diagram describing the steps in a method for operating magnetic body particles. The present invention is concerned with a method for operating magnetic body particles whereby a target substance in a liquid sample is bound to surfaces of the magnetic body particles. Referring to FIG. 1(A), a container 10 includes a liquid sample 31, magnetic body particles 71, and a magnetic solid body 60. The liquid sample 31 contains a target substance to be bound to surfaces of the magnetic body particles 71. The magnetic body particles 71 are particles capable of binding the target substance to the particle surface thereof. The magnetic solid body 60 is a magnetic body having a larger particle diameter than the magnetic body particles 71.

### Container

The material and the shape of the container 10 are not particularly limited, as long as the magnetic solid particles and the magnetic body particles can move inside the container in response to the external magnetic field procedure, and the liquid can be held in the container. For example, the container may be a tubular container such as a test tube, or a conical container such as an Eppendorf tube. The container may have a straight tube structure (capillary) with an inner diameter of about 1 mm to 2 mm, and a length of about 50 mm to 200 mm, or a mated structure of a flat board attached to the top surface of another flat board having a straight groove measuring about 1 mm to 2 mm in width, about 0.5 mm to 1 mm in depth, and about 50 mm to 200 mm in length. The container shape is not limited to tubular or planar, and may be a structure with a branched particle channel of, for example, a cross-shape or a T-shape. When designed as a minimal size container, the container can be used as a microdevice for micro liquid procedures, or a chip for micro liquid procedures.

In the present invention, the magnetic body particles 71 inside the container 10 are movable in the magnetic field procedure, and the container can have a sealed system after the sample is supplied. A sealed-system container can prevent external contamination. This is particularly useful in a procedure for binding of easily degradable substances, such as RNA, to the magnetic body particles. A sealed system may be created by using a method for fusing the opening of the container under heat, or appropriately using various means of sealing. When the particles or the aqueous liquid need to be removed out of the container after the procedure, it is preferable to seal an opening of the container in a removable manner by using, for example, a resin plug.

The material of the container 10 is not particularly limited, as long as it does not block the external magnetic field. Examples of such materials include resin materials such as polyolefins (e.g., polypropylene, and polyethylene), fluoro resins (e.g., tetrafluoroethylene), polyvinyl chloride, polystyrene, polycarbonate, and cyclic polyolefins. It is also possible to use materials such as ceramic, glass, silicone, and metal. The inner wall surface of the container may be coated with, for example, a fluoro resin or silicone to improve water repellency.

A translucent container is preferably used when optical measurements such as measurements of absorbance, fluorescence, chemiluminescence, bioluminescence, and refractive index changes, or photoirradiation are performed during or after the magnetic body particle procedure. The translucent container is also preferable because it allows a user to visually check the progress of the particle procedure taking place inside the container. A non-translucent container, such as a metallic container, is preferably used when the liquid and the magnetic body particles need to be shielded from light. A container with a translucent portion and a non-translucent portion also may be used in certain applications.

### Liquid Sample

The liquid sample 31 contains a target substance of interest for separation and purification. Examples of the target substance include biological substances such as nucleic acids, proteins, sugars, lipids, antibodies, receptors, antigens, ligands, and cells. The liquid sample 31 also contains contaminants, in addition to the target substance. For example, for the separation and purification of nucleic acids from blood, the liquid sample 31 contains a wide range of contaminants, such as proteins and sugars eluted from cells, in addition to the target substance nucleic acids.

The liquid sample 31 is typically a mixture of a biological sample such as blood, and a solution for extracting the target substance from the biological sample. The solution for extracting the target substance may be, for example, a cell lysis solution. The cell lysis solution contains a component capable of lysing cells, such as a chaotropic substance, and a surfactant. Examples of chaotropic salts include guanidine hydrochloride, guanidine isocyanate, potassium iodide, and urea. Chaotropic salts are strong protein denaturants, capable of lysing cell proteins, and liberating the nucleic acids inside the cells nucleus into the liquid. Chaotropic salts also have the effect to inhibit the activity of nucleic acid degrading enzyme. Aside from the foregoing components, the liquid sample 31 may contain various buffers, salts, a variety of auxiliary agents, and organic solvents such as an alcohol.

Extraction of the target substance from a biological sample such as blood typically involves degrading contaminating components in an enzyme reaction to improve the purity and the collection rate of the target substance. For example, for the extraction of nucleic acids from blood, degradation of nuclear proteins bound to nucleic acids is typically performed with a protein degrading enzyme such as protease K. The present invention, on the other hand, performs a magnetic field procedure in the presence of the magnetic body particles and the magnetic solid body, and makes it possible to efficiently and selectively bind the target substance to surfaces of the magnetic body particles, even without an enzyme reaction, as will be described later. It is therefore preferable not to add an enzyme to the liquid sample 31. (This excludes the enzymes inherent to the biological sample.)

### Magnetic Body Particles

The magnetic body particles 71 used in the present invention are particles that are capable of selectively binding the target substance in the liquid sample 31. The binding of the target substance to the particle surface is not particularly limited, and may take place according to known binding mechanisms, including physical bonding, and chemical bonding. For example, the target substance is bound to the surface or inside of the particles via various intermolecular forces such as Van der Waals force, hydrogen bonding, hydrophobic interaction, interionic interaction, and π-π stacking. The binding of target substances such as nucleic acids, proteins, sugars, lipids, antibodies, receptors, antigens, ligands, and cells to the particle surface may take place via molecular recognition. For example, when the target substance is a nucleic acid, selective binding of nucleic acids to the particle surface can be achieved with the use of silica-coated magnetic body particles. When the target substance is an antibody (for example, a labeled antibody), a receptor, an antigen, or a ligand, the target substance can selectively bind to the particle surface via the amino group, carboxyl group, epoxy group, avidin, biotin, digoxigenin, protein A, or protein G on the particle surface.

Examples of the magnetic body include strongly magnetic metals such as iron, cobalt, and nickel, and compounds, oxides, and alloys of such metals. Specific examples include magnetite (Fe₃O₄), hematite (Fe₂O₃, or α-Fe₂O₃), maghemite (γ-Fe₂O₃), titanomagnetite (xFe₂TiO₄.(1-x)Fe₃O₄), ilmenohematite (xFeTiO₃.(1-x)Fe₂O₃), pyrrhotite (Fe₁₋ₓS (x=0 to 0.13)..Fe₇S₈ (x to 0.13)), greigite (Fe₃S₄), goethite (α-FeOOH), chromium oxide (CrO₂), permalloy, an alnico magnet, stainless steel, a samarium magnet, a neodymium magnet, and a barium magnet.

The magnetic body particles have a particle diameter of preferably about 0.1 μm to 20 μm, more preferably about 0.5 μm to 10 μm for ease of particle procedure in the liquid. Preferably, the magnetic body particles are spherical in shape with a uniform particle diameter. However, the magnetic body particles may have an irregular shape with a certain degree of particle size distribution, provided that it enables the particle procedure. The constituent of the magnetic body particles may be a single component, or a plurality of components.

Preferably, the magnetic body particles have surfaces to which a substance for selective binding the target substance is attached or coated with the substance. As such magnetic body particles, commercially available product may be used, for example, from Life Technologies under the trade name Dynabeads®, or from. Toyobo under the trade name MagExtractor®.

### Magnetic Solid Body

The material of the magnetic solid body 60 used in the present invention is not particularly limited, as long as it is a magnetic body. Examples of such materials include strongly magnetic metals such as iron, cobalt, and nickel, and compounds, oxides, and alloys of such metals, as with the case of the magnetic body exemplified for the magnetic body particles. The shape of the magnetic solid body is not particularly limited, and may be, for example, spherical, polyhedral, flat, or rod-like.

Preferably, the magnetic solid body has a larger particle diameter than the magnetic body particles. The major axis of the magnetic solid body is regarded as the particle diameter when the magnetic solid body is non-spherical. The particle diameter of the magnetic solid body is preferably 100 μm or more, more preferably 300 μm or more, further preferably 500 μm or more. By the presence of the magnetic solid body having a larger particle diameter, the magnetic field procedure can move and disperse the magnetic body particles in the liquid, even when the magnetic body particles are forming aggregates. The particle diameter of the magnetic solid body is preferably at least 10 times, more preferably at least 20 times, further preferably at least 30 times, particularly preferably at least 50 times larger than the particle diameter of the magnetic body particles.

The upper limit of the particle diameter of the magnetic solid body is not particularly limited, as long as the magnetic solid body can move inside the container. For example, a spherical magnetic solid body may have a particle diameter smaller than the inner diameter of the container when the container is tubular. The particle diameter of the magnetic solid body is preferably 10 mm or less, more preferably 5 mm or less, further preferably 3 mm or less, particularly preferably 1.5 mm or less for ease of magnetic field procedure. The particle diameter of the magnetic solid body is preferably at most 100,000 times, more preferably at most 50,000 times, further preferably at most 10,000 times the particle diameter of the magnetic body particles. The embodiment represented in FIG. 1 uses a single magnetic solid body 60 in the container 10. It is, however, possible to use more than one magnetic solid body.

Commercially available metal balls such as iron balls and stainless steel balls for ball bearings may be directly used as the magnetic solid body. The magnetic solid body may have functionality. For example, the surface of a metallic material such as iron and stainless steel may be coated to provide corrosion resistance against the reagents or the sample.

The iron or other materials of the magnetic solid body easily become corroded, and the corroded component (for example, the metal ions eluted in the liquid layer) may interfere with the binding of the target substance, or with the subsequent reaction with the reagents or the sample (for example, an enzyme reaction, and an antigen-antibody reaction), or the elution of the target substance, particularly when magnetic solid body is in contact with the aqueous liquid in the particle operating device for extended time periods. Such adverse effects of metal corrosion can be reduced when the magnetic solid body has a coating layer on the metal surface to prevent corrosion.

When the metal surface is coated to provide corrosion resistance, the coating material is not particularly limited, as long as it can prevent metal corrosion in the gelatinous medium or in the liquid layer. The coating material may be an inorganic material such as metals and metal oxides, or a resin material. Examples of the metallic material include gold, titanium, and platinum. Examples of the resin material include fluoro resins such as tetrafluoroethylene, and epoxy resins. A preferred coating material is one that has a small inhibitory effect on the reaction with the reagents or the sample, or that has only a small influence on the binding and elution of the sample.

The method used to form a coating layer on the metal surface is not particularly limited. For example, methods such as plating, and drying (e.g., vapor deposition, sputtering, and CVD) are preferably used when a metal coating of, for example, gold, titanium, or platinum is formed to make the metal surface corrosion resistant. Wet coating is preferably used when coating the metal surface with a resin.

The metal may become exposed, and corroded at the exposed portion when the coating provided to prevent metal corrosion becomes detached or scratched by physical impact or the like. To prevent this, the thickness of the coating layer is preferably several micrometers to several hundreds of micrometers. Preferably, a coating layer of such a thickness range is formed by forming a resin layer by wet coating. The resin material may be, for example, a resin solution, or a liquid adhesive. As the liquid adhesive, a commercially available product intended for metals may be directly used. For example, a two-component curable epoxy-based adhesive, which is curable at ordinary temperature, can easily form a coating layer of the foregoing thickness, and is preferred for use as the coating material for preventing metal corrosion.

When drying and curing a resin solution by wet coating, it is preferable to set drying conditions so that the coating layer will not be detached. For example, when drying or curing the coated magnetic solid body while allowing it stand, it is preferable to place the coated magnetic solid body on a material to which the resin material is hard to adhere, or on a material having solvent resistance against the solvent of the coating solution.

The surface of the magnetic solid body may have a coating layer other than the coating layer provided against corrosion. For example, the surface of the magnetic solid body may be coated with various functional molecules so that substances different from the substance that binds to the magnetic body particles bind to the surface of the magnetic solid body. It is also possible to coat the surface of the magnetic solid body with an optical material such as a luminescent material, and a fluorescent material. Such a configuration enables optical detection of the magnetic solid body's location, and is applicable for, for example, the detection or correction of the location of the magnetic solid body or magnetic body particles in automating the particle procedure. The magnetic solid body also can function as an actuator for the valve and pump operations of the magnetic field procedure in a microchannel system when the material, the size, and the shape of the magnetic solid body are adjusted. The magnetic solid body also may be used as a receptor of the driving power of a magnetic resonance fluid control device, or as a heated body of electromagnetic induction to provide a heat source of chemical reaction.

### Lysis and Binding of Target Substance by Magnetic Field Procedure

The following primarily describes an example of binding nucleic acids as a target substance to surfaces of the magnetic body particles, with reference to FIGS. 1(A) to (C). As shown in FIG. 1(A), the liquid sample 31, the magnetic body particles 71, and the magnetic solid body 60 are loaded into the container 10. The loading order is not particularly limited.

When the target substance is a nucleic acid, the liquid sample 31 contains biological samples such as an animal or plant tissue, a bodily fluid, and wastes, or other nucleic acid-containing materials such as cells, protozoa, fungi, bacteria, and viruses. The bodily fluid includes blood, spinal fluid, saliva, and milk, whereas the wastes include feces, urine, and sweat. Two or more of them may be used in combination. The cells include white blood cells and platelets in the blood, exfoliated cells of mucosal cells such as buccal cells, and white blood cells in saliva. These may be used in combination. A liquid sample containing nucleic acids may be prepared in the form of, for example, a mixture with a cell suspension, a homogenate, and a cell lysis solution. The liquid sample also may be prepared by adding blood or other samples to a container 10 that has been loaded with a solution such as a cell lysis solution. Conversely, a cell lysis solution may be injected into a container 10 that has been loaded with blood or other samples.

Blood or other samples may be added to a container 10 that has been loaded with the magnetic particles, the magnetic solid body, and a cell lysis solution. A container 10 loaded with the magnetic body particles, the magnetic solid body, and a cell lysis solution may be prepared as a kit. The present invention does not require an enzyme treatment of the sample, and enables binding of nucleic acids to the magnetic body particle surface with the simple procedure of supplying blood or other samples to a stored container that has been loaded with the magnetic body particles, the magnetic solid body, and a cell lysis solution. Preferably, the container 10 loaded with the liquid sample, the magnetic body particles, and the magnetic solid body is closed at the top of the container 10 to create a sealed system in the device, and prevent external contamination.

The target substance nucleic acid can be bound to the magnetic body particle surface (silica coating) by sufficiently dispersing the magnetic body particles in the container 10 loaded with the liquid sample 31, the magnetic body particles 71, and the magnetic solid body 60. This is achieved by the magnetic field procedure performed from outside the container. As shown in FIG. 1(B), the magnetic solid body 60 and the magnetic body particles 71 become attracted toward the inner wall surface of the container in an area around a magnet 9 brought closer the outer wall surface of the container. The magnetic field procedure may use a magnetic source such as a permanent magnet (for example, a ferrite magnet, and a neodymium magnet), and an electromagnet.

The liquid sample 31 contains contaminants originating in the sample. Among such contaminants, denatured proteins have a function to mask the surfaces of the magnetic body particles, and bind the magnetic body particles to each other. This may cause the magnetic body particles 71 attracted to the inner wall surface of the container to form aggregates, and reduce the opportunity for nucleic acids in the liquid sample to contact the magnetic body particles, inhibiting the binding of the target substance to the particle surface.

In the present invention, the magnetic field procedure moves the magnetic body particles with the magnetic solid body in the container containing the magnetic body particles 71 and the magnetic solid body 60. Specifically, the magnetic field procedure is a procedure that moves the magnet 9. The magnet motion may be, for example, a linear motion including a reciprocating motion, or a rotational motion, or any other motions including a motion that involves an irregular trajectory. The magnetic field procedure causes aggregates of magnetic body particles to disperse in the liquid sample, as shown in FIG. 1(C). For efficient dispersion of the magnetic body particles, it is preferable to move the magnet 9 in a reciprocating motion along the outer wall surface of the container 10.

The underlying principle by which the magnetic body particles become dispersed as they move with the magnetic solid body in the liquid remains somewhat unclear. However, observations of the magnetic solid body and magnetic body particle movement have confirmed that small vibrations occur in the magnetic solid body 60 as it moves along the inner wall surface of the container 10, due to frictional resistance with the inner wall surface of the container, or a delay in following the magnet movement. It is presumed that such small vibrations of the magnetic solid body act to disperse the magnetic body particles around the magnetic solid body, or break up aggregates of magnetic body particles that are present between the container wall surface and the magnetic solid body, and cause the magnetic body particles to quickly disperse in the liquid.

The magnetic field procedure performed in the presence of the magnetic body particles and the magnetic solid body breaks the aggregation state of the magnetic body particles, and disperses the magnetic body particles. This increases the opportunity for the magnetic body particles to contact the target substance in the liquid sample, and allows the target substance to selectively bind to the surfaces of the magnetic body particles. By allowing the target substance to selectively bind to the surfaces of the magnetic body particles, the method enables efficient collection of the target substance with high purity. The present invention can thus eliminate the need for an enzyme treatment for the lysis and binding of a sample in the separation and purification of a target substance with magnetic body particles.

Because an enzyme treatment is not required, the cost of the separation and purification procedure can be reduced. Further, because addition of an enzyme is not necessary, there is no need to add samples or perform a dispensing procedure. This simplifies the procedure, and reduces the risk of contamination. The risk of contamination also becomes smaller with the method of the present invention because the method can be performed in a sealed system, unlike the dispersion procedure by pipetting required to perform in the open system. It is also possible to easily achieve automation with the method of the present invention because the method enables dispersing the magnetic body particles in the liquid with a procedure as simple as moving a magnet in a reciprocating motion.

### Post-Lysis/Binding Procedure

The magnetic body particles 71 with the bound target substance are separated from the liquid sample 31, and sent to the subsequent steps. For example, in the separation and purification of nucleic acids, the magnetic body particles 71 are washed in a washing liquid to remove the contaminants attached to the surface, and the target substance nucleic acids binding to the magnetic body particles are eluted and liberated in an elution liquid, and collected. The collected nucleic acids may be subjected to procedures such as concentration, and solidification, as required, and used for analysis or reaction, or other purposes.

The washing and elution procedures may be performed by using known methods. For example, for washing and elution, the liquid inside the container may be removed while the magnetic body particles are being magnetically immobilized in the container in the vicinity of a magnet brought close to the container, and the magnetic body particles may be dispersed in the liquid after supplying a new liquid (a washing liquid or an elution liquid) to the container. The magnetic body particles may be dispersed in the liquid by using techniques such as pipetting and stirring (e.g., vortexing), or using the magnetic field procedure. Here, the magnetic solid body may be taken out of the container, or kept in the container with the magnetic body particles.

The foregoing example primarily described separation and purification of nucleic acids with the magnetic body particles. However, the target substance bound to the magnetic body particles is not limited to nucleic acids, and the present invention is also applicable to various target substances other than nucleic acids. For example, with the magnetic field procedure performed in the presence of the magnetic body particles and the magnetic solid body, target substance antibodies may be selectively bound to surfaces of magnetic body particles coated with molecules that can selectively bind to antibodies such as protein G and protein A. An enzyme immunoassay (ELISA: enzyme-linked immuno-sorbent assay) is possible by contacting antibody-bound magnetic body particles to a liquid containing analyte antigens, and to enzyme labeled secondary antibodies, and monitoring the chromogenic reaction between a chromogenic substance and the enzyme linked to the secondary antibodies bound to the magnetic body particle surface.

By changing the liquid loaded into the device according to the types of target substances, or the intended procedure, the present invention is applicable not only to extraction, purification, and separation of a target substance, but to a variety of other applications, including various reactions, detections, and qualitative and quantitative analyses.

Procedure with Device Using Sealed Gelatinous Medium

The method of the present invention is also applicable to separation and purification of a target substance using a device in which aqueous liquid layers and gelatinous medium layers are alternately disposed as in the device disclosed in PTL 2 (WO2012/086243). Such a device enables a series of procedures to be performed in a sealed system, and involves a reduced contamination risk compared to the pipetting procedure performed in an open system.

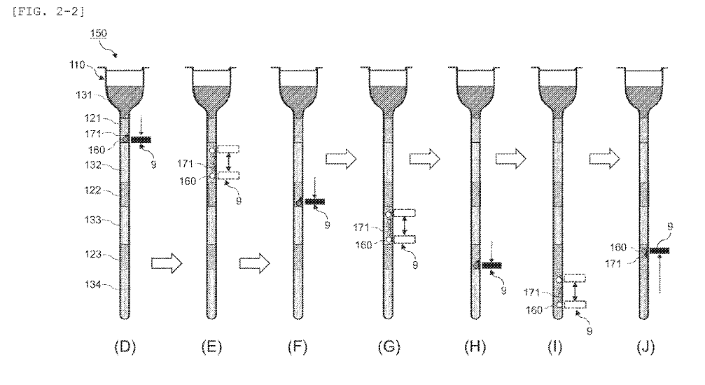

The following describes an example of the separation and purification of nucleic acids with a device including alternately disposed aqueous liquid layers and gelatinous medium layers, with reference to FIG. 2. FIG. 2-1(A) illustrates a tubular device 150 in which a nucleic acid extraction liquid 130, a first washing liquid 132, a second washing liquid 133, and a nucleic acid elution liquid 134 are loaded into a tubular container 110 via gelatinous medium layers 121, 122, and 123 along the direction of movement of magnetic body particles 171.

The gelatinous medium forming the gelatinous medium layers 121, 122, and 123 is not limited, as long as it is gel-like or paste-like before the particle procedure. Preferably, the gelatinous medium is a substance that is insoluble or poorly soluble in the adjacent liquid layers, and is chemically inert. When the liquid layers are aqueous liquid layers, the gelatinous medium is preferably an oil gel that is insoluble or poorly soluble in the aqueous liquid. It is also preferable that the gelatinous medium layers are layers of a chemically inert substance. As used herein, “insoluble or poorly soluble in the liquid” means that the solubility in the liquid at 25° C. is about 100 ppm or less. A chemically inert substance refers to a substance that does not chemically interfere with the liquid layers, the magnetic body particles, or the substance bound to the magnetic body particles even in contact with the liquid layers or in the magnetic body particle procedure (specifically, the procedure with which the magnetic body particles are moved in the gelatinous medium).

The composition or properties of the gelatinous medium are not particularly limited. The gelatinous medium is formed by, for example, adding a gelatinizer to a water-insoluble or poorly water-soluble liquid materials such as a liquid oil or fat, an ester oil, a hydrocarbon oil, and a silicone oil, and forming a gel. The gel (physical gel) formed by addition of the gelatinizer has a three-dimensional network held by weak intermolecular bonds such as hydrogen bonding, Van der Waals force, hydrophobic interaction, and electrostatic attraction, and undergoes a reversible solgel transition in response to external stimuli such as heat. Examples of the gelatinizer include hydroxyfatty acids, dextrin fatty acid esters, and glycerin fatty acid esters. The gelatinizer is used in an amount that is appropriately decided according to factors such as the physical properties of the gel. For example, the gelatinizer is used in an amount of 0.1 to 5 weight parts with respect to 100 weight parts of the water-insoluble or poorly water-soluble liquid material.

The gelation method is not particularly limited. For example, a water-insoluble or poorly water-soluble liquid material is heated, and a gelatinizer is added to the heated liquid material. A physical gel forms upon completely dissolving the gelatinizer, and cooling the mixture to a temperature equal to or less than the solgel transition temperature. The heating temperature is appropriately decided according to the physical properties of the liquid material and the gelatinizer.

The gelatinous medium may be one prepared by equilibrium swelling of a hydrogel material (for example, gelatin, collagen, starch, pectin, hyaluronan, chitin, chitosan, alginic acid, and derivatives thereof) with liquid. For example, the hydrogel may be one obtained through chemical crosslinkage of a hydrogel material, or a gel formed with a gelatinizer (for example, salts of alkali metals or alkali earth metals such as lithium, potassium, and magnesium; salts of transition metals such as titanium, gold, silver, and platinum; silica, carbon, or alumina compounds).

The gelatinous medium and the liquid may be loaded into the container 110 by using an appropriate method. When the container is a tubular container, it is preferable to first seal the opening at one end of the container before loading, and load the gelatinous medium and the aqueous liquid one after another from the other opening. For loading into a small structure such as a capillary with an inner diameter of about 1 to 2 mm, for example, the gelatinous medium is loaded by being pushed into the predetermined position of the capillary with a metallic injection needle attached to a lure lock syringe.

The volume of the gelatinous medium and the liquid loaded into the container may be appropriately set according to such factors as the amount of the magnetic body particles used for the procedure, and the type of the procedure. When providing more than one gelatinous medium layer and liquid layer in the container, the volume of each layer may be the same or different. The layer thickness may be appropriately selected, and is preferably, for example, about 2 mm to 20 mm when factors such as operability are considered.

The nucleic acid extraction liquid 130 used for extraction of nucleic acids may be, for example, the cell lysis solution described above (for example, a chaotropic substance, chelating agents such as EDTA, and buffers containing trishydrochloric acid). In the uppermost part of the container 110 are the nucleic acid extraction liquid 130, and magnetic body particles 171 and a magnetic solid body 160, which are loaded in advance. The magnetic body particles 171 are particles that are capable of selectively binding nucleic acids. For example, silica-coated magnetic body particles are used.

A nucleic acid-containing sample, such as blood, is added to the nucleic acid extraction liquid 130 through the upper opening of the device 150 containing the alternately disposed liquid layers and gelatinous medium layers. This produces a solution (liquid sample) 131 of a nucleic acid extraction liquid and nucleic acids. The magnetic solid body 160, and the magnetic body particles 171 become attracted toward the inner wall surface of the container in an area around the magnet 9 brought closer to the side surface of the container containing the liquid sample 131 (FIG. 2-1(B)). Moving the magnet 9 in a reciprocating motion along the outer wall surface of the container 110 causes the magnetic solid body 160 to move in the liquid sample, and the magnetic body particles 171 become dispersed in the liquid sample 131 along with the movement of the magnetic solid body 160 (FIG. 2-1(C)). As a result of this procedure, the nucleic acids in the liquid sample selectively bind to the surfaces of the magnetic body particles.

The subsequent steps may be performed after taking the magnetic solid body 160 out of the system, or with the magnetic solid body 160 kept in the system with the magnetic body particles 171. The washing and elution efficiencies can improve when the magnetic solid body 160 and the magnetic body particles 171 are moved in the device without removing the magnetic solid body 160. With the magnetic solid body 160 kept in the system, the device can remain sealed, and the contamination risk can be reduced.

Moving the magnet 9 along the outer wall surface of the container causes the magnetic body particles 171 to move into the gelatinous medium layer 121. Here, the magnetic body particles 171 move in the gelatinous medium integrally with the magnetic solid body 160 (FIG. 2-2(D)). In entry of the magnetic body particles 171 to the gelatinous medium layer 121, most of the liquid physically held around the magnetic body particles 171 in the form of droplets desorbs from the particle surface, and remain in liquid in the liquid layer 131. On the other hand, the magnetic body particles 171 can easily move into the gelatinous medium layer 121 with the target substance bound to the particles.

The entry and the movement of the magnetic body particles 171 and the magnetic solid body 160 in the gelatinous medium layer 121 creates pores in the gelatinous medium. However, the gel repairs itself with its thixotropic property. When a shear force is applied while the magnetic body particles are moving in the gel in the magnetic field procedure, the thixotropic property of the gel causes the gel to locally fluidize (become less viscous). This allows the magnetic body particles and the magnetic solid body to easily move through the gel by piercing through the fluidized portions. The gel liberated from the shear force following the passage of the magnetic body particles quickly restores the original elastic state. Accordingly, no pores remain after the passage of the magnetic body particles, and hardly any liquid flows into the gel through the pierced portions created by the magnetic body particles. The gel may be physically destroyed, and may lose its ability to restore itself when the magnet 9 is moved at excessively high speeds. To prevent this, the magnet is moved at a rate of preferably about 0.1 to 5 mm/s.

The restoring of the gel due to the thixotropic property acts to squeeze the liquid carried by the magnetic body particles 171. This makes it possible to separate the magnetic body particles and the liquid droplets upon restoration of the gel even when the magnetic body particles 171 form aggregates, and move into the gelatinous medium layer 121 with the liquid droplets trapped in the aggregates.

The magnetic field procedure moves the magnetic body particles 171 and the magnetic solid body 160, which have passed through the gelatinous medium layer 121, from the gelatinous medium layer 121 to the liquid layer 132. As described above, the passage of the magnetic body particles and the magnetic solid body through the gelatinous medium layer 121 does not leave pores, and there is hardly any flow of the liquid sample 131 into the liquid layer 132.

The liquid layer 132 is a washing liquid, for example. The washing liquid is not limited, as long as it can liberate non-nucleic acid components attached to the magnetic body particles (for example, proteins, and carbohydrates), and the reagents used for the process (for example, the nucleic acid extraction liquid) into the washing liquid while allowing the nucleic acids to remain bound to the surfaces of the magnetic body particles. Examples of the washing liquid include high-salt-concentration aqueous solutions of, for example, sodium chloride, potassium chloride, or ammonium sulfate; and aqueous solutions of alcohols such as ethanol, and isopropanol. The liquid layer 133 also may be a washing liquid. When the liquid layers 132 and the 133 are both washing liquids, the washing liquids may have the same or different compositions.

Moving the magnet 9 along the side surface of the liquid layer 132 causes the magnetic solid body 160 and the magnetic body particles 171 to move in the liquid layer along with the movement of the magnet 9. Here, the magnetic body particles 171 forming aggregates become dispersed in the liquid layer 132 (FIG. 2-2(E)). By moving the magnetic body particles 171 in the liquid layer with the magnetic solid body 160, it is possible to efficiently disperse the magnetic body particles in the liquid, and to improve the washing efficiency, as with the case of the lysis and binding. For improved washing efficiency, it is preferable to move the magnet in a reciprocating motion along the side surface of the liquid layer 132 (outer wall surface of the container).

The magnet 9 is then moved from the side surface of the liquid layer 132 to the side surface of the gelatinous medium layer 122 (FIG. 2-2(F)). After further moving the magnet 9 down to the side surface of the liquid layer 133, the magnet 9 is moved in a reciprocating motion to sufficiently disperse the magnetic body particles, and wash the magnetic body particles in the liquid layer 133 (FIG. 2-2(G)).

In the example represented in FIG. 2, two washing liquid layers, 132 and 133, are loaded in the container 110 via the gelatinous medium layer 122. However, only one, or three or more washing liquid layers may be used instead. It is also possible to omit the washing procedure, provided that it does not unfavorably interfere with the purpose of separation, or the intended use.

The magnet 9 is moved from the side surface of the second washing liquid 133 to the side surface of the gelatinous medium layer 123 to move the magnetic body particles 171 and the magnetic solid body 160 into the gelatinous medium layer 123 (FIG. 2-2(H)). By further moving the magnet 9 down to the side surface of the nucleic acid elution liquid 134, the magnetic body particles 171 and the magnetic solid body 160 move into the nucleic acid elution liquid 134.

The nucleic acid elution liquid may be water, or a buffer containing a low concentration of salt. Specifically, nucleic acid elution liquid may be, for example, a Tris buffer, a phosphate buffer, or distilled water. Typically, the nucleic acid elution liquid is a 5 to 20 mM Tris buffer with an adjusted pH of 7 to 9. The nucleic acids bound to the surfaces of the magnetic body particles become liberated as the particles with the nucleic acids bound to the particle surface thereof move into the nucleic acid elution liquid. Specifically, the liberation of the nucleic acids may take place, for example, through dispersion of the particles in the elution liquid. For example, moving the magnet 9 along the side surface of the nucleic acid elution liquid 134 moves the magnetic body particles 171 with the magnetic solid body 160, and the magnetic body particles 171 become dispersed in the nucleic acid elution liquid (FIG. 2-2(I)). As a result, the nucleic acids bound to the surfaces of the magnetic body particles 171 become efficiently desorbed and liberated in the nucleic acid elution liquid. This improves the nucleic acid collection rate.

The magnet 9 is then moved toward the gelatinous medium layer 123 along the outer wall surface of the container, as required, to send the magnetic body particles 171 and the magnetic solid body 160 back into the gelatinous medium layer 123, as shown in FIG. 2-2(J). This procedure removes the magnetic body particles 171 and the magnetic solid body 160 from the nucleic acid elution liquid 134, and makes it easier to collect the nucleic acid elution liquid.

As described above, in a case where a device including alternately disposed liquid layers and gelatinous medium layers is used, the liquid layer does not allow external access because the device is a sealed system containing the liquid layer between the gelatinous medium layers or between the gelatinous medium layer and the container. In the embodiment of the invention, however, the magnetic body particles can be dispersed in the liquid layer while retaining the sealed system, and the risk of external contamination can be reduced compared to when the magnetic body particles are dispersed by a pipetting procedure.

In the embodiment, the magnetic body particles are moved in the gelatinous medium layer to effect solid-liquid separation. This makes it possible to more efficiently separate and collect a target substance with smaller amounts of magnetic body particles or reagents, and more effectively reduce the amount of waste fluid than when the solid-liquid separation of magnetic body particles from reagents such as the washing liquid and the elution liquid is performed by a pipetting procedure. It is also easy to automate the procedures because the procedure from the binding to the elution of a target substance can be performed by simply moving the magnet along the outer wall surface of the container after adding a sample (e.g., blood) to the lysis/binding liquid (nucleic acid extraction liquid).

### Device and Kit for Particle Procedure

The method of the present invention does not require an enzyme treatment for lysis and binding of a target substance, and a device to be used for the procedure can be easily produced. Specifically, the device for lysis and binding shown in FIG. 1 can be produced simply by loading the magnetic body particles, the magnetic solid body and a liquid in a container. The liquid loaded into the container is, for example, a liquid capable of lysing cells (e.g., nucleic acid extraction liquid). Additives such as an alcohol may be added to the liquid to prevent aggregation of the magnetic body particles.

The magnetic body particles, the magnetic solid body, and the liquid may be independently provided, separately from the container. For example, the magnetic body particles may be independently provided either by itself or by being dispersed in a liquid, separately from the body of the device containing a container that has been loaded with the magnetic solid body, and the liquid capable of lysing cells. In this case, the magnetic body particles may be provided as a component of a kit for making the device. The magnetic solid body also may be separately provided from the body of the device. A liquid containing the magnetic body particles and the magnetic solid body may be provided as a kit component. When the device or a kit is provided to include the magnetic solid body being dispersed in a liquid, the magnetic solid body remains in contact with the liquid for a long period of time depending on storage conditions of the device or the kit before use. In order to prevent corrosion or deterioration of the magnetic solid body, the metal surface of the magnetic solid body is preferably coated, as described above.

The device with alternately disposed liquid layers and gelatinous medium layer shown in FIG. 2 also can be produced with ease. The gelatinous medium and the liquid may be loaded into the container immediately before the particle procedure, or well in advance of the particle procedure. A reaction or absorption hardly occurs between the gelatinous medium and the liquid even after long hours from the loading when the gelatinous medium is insoluble or poorly soluble in the liquid, as described above.

The device with alternately disposed liquid layers and gelatinous medium layers for use in the magnetic body particle procedure may be provided to include a container loaded with the magnetic body particles 171 and the magnetic solid body 160, as shown in FIG. 2-1(A). The magnetic solid body 160, shown as being loaded in the liquid layer 130 in FIG. 2-1(A), may be loaded in, for example, the gelatinous medium layer 121 instead. In this case, the magnetic solid body 160 in the gelatinous medium layer 121 may be moved into the liquid layer with the magnetic field procedure before operating the magnetic body particles for lysis and binding.

The amount of the magnetic body particles contained in the device or the kit is appropriately decided according to factors such as the type of the chemical procedure involved, and the volume of each liquid layer. Typically, the magnetic body particles are used in an amount of preferably, for example, about 10 to 200 μg when the container is a narrow cylindrical capillary having an inner diameter of about 1 to 2 mm.

### EXAMPLES

The present invention is described below in detail through exemplary experiments conducted to extract DNA from human whole blood using silica-coated magnetic beads. It is to be noted that the present invention is not limited by the following examples.

### Reference Example 1

### Elution/Binding

Human whole blood (200 μL) was collected into a 1.5-mL polypropylene tube (Eppendorf Safe-Lock Tube, Cat. No. 0030 120.086), and 5 μL, of a proteinase K aqueous solution (20 mg/mL) was added. After mixing these components for 10 seconds, 100 μL of a lysis/binding liquid (30 mM Tris-HCl, pH 8.0, 30 mM EDTA, 5% Tween-20, 0.5% Triton X-100, and 800 mM guanidine hydrochloride) was added, and mixed for 10 seconds. The mixture was incubated for 5 minutes in an aluminum block thermostat bath that had been heated to 68° C. Immediately after taking out the tube from the thermostat bath, 1 mg of magnetic beads suspended in 75 μL of isopropanol (silica-coated magnetic beads for nucleic acid extraction appended to the nucleic acid extraction kit available from Toyobo under the trade name MagExtractor™-Genome; average particle size of about 3 μm) was added, and the mixture was stirred for 5 minutes with a vortex mixer equipped with an adapter for continuous stirring. The tube was then set on a magnetic body particle separation stand, and the liquid was removed from the tube on the stand with a micropipette after being allowed to stand for 1 minute.

### Washing

The tube was removed from the stand, and 500 μL of a first washing liquid (37% ethanol, 4.8 M guanidine hydrochloride, 20 mM Tris-HCl, pH 7.4) was added. After pipetting and thoroughly resuspending a mass of magnetic beads that have collected on the inner wall of the tube, the tube was again set on the stand, and allowed to stand for 1 minute. The liquid was then removed from the tube on the stand with a micropipette. After removing the tube from the stand, 500 μL of a second washing liquid (2 mM Tris-HCl, pH 7.6, 80% ethanol, 20 mM NaCl) was added, and the liquid was removed from the tube in the same fashion as described above after resuspending the particles and setting the tube on the stand.

### Elution

The tube was removed from the stand, and 200 μL of distilled water was added as an elution liquid. A mass of magnetic beads was pipetted and resuspended, and allowed to stand for 5 minutes at room temperature. After pipetting and resuspending the magnetic beads, the tube was set on the stand, and the liquid (DNA eluted liquid) in the tube on the stand was collected with a micropipette after being allowed to stand for 1 minute on the stand.

### Example 1

Human whole blood (200 μL) was collected into a 1.5-mL polypropylene resin tube, and a lysis/binding liquid (50 mM Tris-HCl, pH 6.4, 10% Triton X-100, 4 M guanidine isocyanate) was added without proteinase K. These were mixed for 10 seconds. A suspension of magnetic beads in isopropanol was then added to the mixture as in Reference Example 1, and a steel ball having a particle diameter of 1 mm (available from Shintokogio Ltd.) was added. Thereafter, a neodymium magnet (a columnar magnet measuring 6 mm in diameter, and 23 mm in length, available from Niroku Seisakusho Co., Ltd. under the trade name NE127) was moved back and forth at a rate of 5 times per second along the outer wall surface of the tube over an about 2-cm distance from the bottom to the cap portion of the tube. Here, the magnet was moved so that the steel ball was able to follow the magnet movement. Observation of the tube confirmed that the magnetic beads were dispersed in the liquid.

The tube was set on a magnetic body particle separation stand, and the liquid was removed from the tube on the stand with a micropipette after being allowed to stand for 1 minute. This was followed by washing, elution, and collection of the DNA eluted liquid as in Reference Example 1.

### Comparative Example 1

A lysis/binding liquid, and a suspension of magnetic beads in isopropanol were added to human whole blood, as in Example 1. The mixture was stirred with a vortex mixer for 1 minute without adding a steel ball, and the liquid was removed from the tube set on a magnetic body particle separation stand. This was followed by washing, elution, and collection of the DNA eluted liquid as in Reference Example 1.

### Evaluation

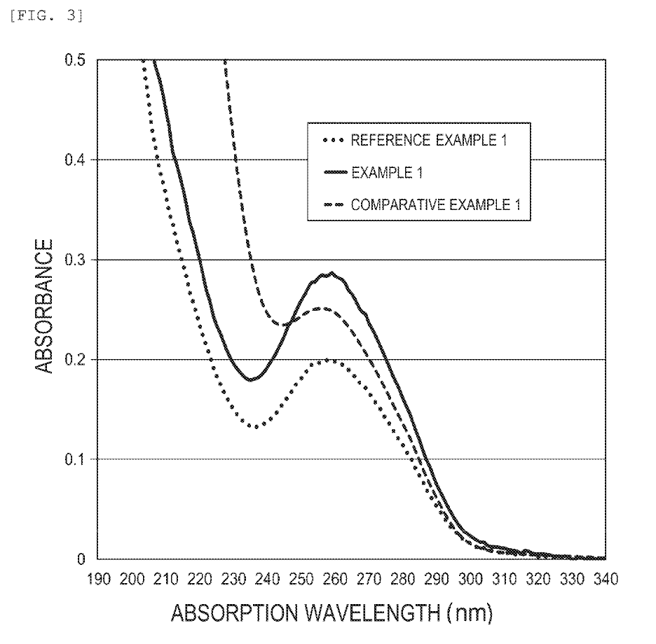

The UV absorption spectra of the elution liquids collected in Reference Example, Example, and Comparative Example were measured with a spectrophotometer (BioSpec nano available from Shimadzu Corporation). The results are shown in FIG. 3. Absorbance ratios at 230 nm, 260 nm, and 280 nm wavelengths (A₂₆₀/A₂₈₀, and A₂₆₀/A₂₃₀) were determined from the UV absorption spectra. The results are presented in Table 1 along with the collected DNA amounts.

TABLE 1

| | Lysis/adsorption | | | | |
|---|---|---|---|---|---|
| | conditions | | DNA purity and collected amount | | |
| | Enzyme | Metal | | | Collected |
| | treatment | ball | A₂₆₀/A₂₈₀ | A₂₆₀/A₂₃₀ | amount (μg) |
| Reference | Present | Absent | 1.735 | 1.322 | 3.27 |
| Ex. 1 | | | | | |
| Example 1 | Absent | Present | 1.761 | 1.449 | 4.68 |
| Comparative | Absent | Absent | 1.691 | 0.399 | — |
| Ex. 1 | | | | | |

A peak minimum (peak trough) of absorbance occurs in the vicinity of 230 nm in high purity DNA, and larger absorbance ratios at 260 nm and 230 nm (A₂₆₀/A₂₃₀) mean higher purity. An absorbance minimum occurred in the vicinity of 230 nm, and DNA purification was confirmed in Reference Example 1 in which an enzyme treatment was performed for lysis and binding, and in Example 1 in which the lysis and binding involved the procedure with the magnetic body particles in the presence of the steel ball. On the other hand, the absorbance minimum shifted toward the vicinity of 240 nm, and the A₂₆₀/A₂₃₀ ratio had a smaller value of about 0.4 in Comparative Example 1 in which the lysis and binding did not involve an enzyme treatment. Presumably, this is the result of increased background absorption on the shorter wavelength side due to the inclusion of large numbers of low-molecular contaminating components. It was accordingly not possible to accurately quantify the amount of collected DNA in Comparative Example 1.

Reference Example 1 had an A₂₆₀/A₂₃₀ ratio of about 1.3, an acceptable level of purification for PCR and similar applications. However, the amount of collected DNA was not sufficient in Reference Example 1. It can be inferred from this result that stirring with a vortex mixer is not sufficient to overcome the masking of the magnetic bead surface by peptides or other contaminants even when the lysis and binding involves an enzyme treatment, and that such masking inhibits binding of DNA to the magnetic bead surface, and lowers the purity of DNA.

On the other hand, the A₂₆₀/A₂₃₀ ratio was higher than 1.4 in Example 1, despite the absence of an enzyme treatment. The absorbance ratio at 260 nm and 280 nm (A₂₆₀/A₂₈₀) as an index of DNA purity was also higher than in Reference Example 1. It can be seen from these results that Example 1 is more desirable in both DNA purity and DNA amount than Reference Example 1 involving an enzyme treatment. As demonstrated by these results, selective binding of a target substance to the magnetic bead surface is possible, and the target substance can be obtained in high purity with the magnetic field procedure performed in the presence of the magnetic solid body having a larger particle diameter than the magnetic beads, even when the lysis and binding procedure does not involve an enzyme treatment.

### REFERENCE SIGNS LIST

10, 110: Container
60, 160: Magnetic solid body
71, 171: Magnetic body particles
31, 131: Liquid sample
9: Magnet
150: Device for operating particles for nucleic acid extraction
121 to 123: Gelatinous medium
130: Liquid layer (nucleic acid extraction liquid)
132, 133: Liquid layer (washing liquid)
134: Liquid layer (nucleic acid elution liquid)

1-11. (canceled)

12. A method for operating magnetic body particles whereby a target substance in a liquid sample is bound to surfaces of the magnetic body particles, the magnetic body particles being particles capable of selectively binding the target substance, the method comprising:
the magnetic body particles being dispersed in the liquid sample to selectively bind the target substance to surfaces of the magnetic body particles with a magnetic field procedure performed from outside a container in the presence of the liquid sample, the magnetic body particles, and a magnetic solid body having a larger particle diameter than the magnetic body particles in the container to move the magnetic body particles with the magnetic solid body along an inner wall surface of the container in the liquid sample.

13. The method according to claim 12, wherein the target substance which the magnetic body particles are capable of selectively binding is at least one selected from the group consisting of nucleic acids, proteins, sugars, lipids, antibodies, receptors, antigens, ligands, and cells.

14. The method according to claim 12, wherein the liquid sample contains a component capable of lysing a cell.

15. The method according to claim 12, wherein the magnetic solid body has a particle diameter of 100 μm or more.

16. The method according to claim 12, wherein the magnetic solid body has a particle diameter that is at least 10 times larger than the particle diameter of the magnetic body particles.

17. The method according to claim 12, wherein the magnetic field procedure moves the magnetic body particles with the magnetic solid body in a reciprocating motion in the liquid sample.

18. The method according to claim 12, wherein the magnetic solid body has a surface with a coating layer for preventing corrosion in the liquid.

19. A method for operating magnetic body particles, comprising:
selectively binding a target substance to surfaces of magnetic body particles according to the method of claim 12; and
contacting the magnetic body particles having the target substance bound thereto to an elution liquid to elute the target substance in the elution liquid.
